(19) United States
(12) Patent Application Publication
(10) Pub. No.: US 2024/0428950 A1
(43) Pub. Date: Dec. 26, 2024

(54) METHOD AND SYSTEM FOR SEMANTIC UNDERSTANDING OF SUBJECTIVE HEALTH

(71) Applicant: Adhera Health, Inc., Santa Cruz, CA (US)

(72) Inventors: Ricardo C. BERRIOS, Santa Cruz, CA (US); Luis FERNANDEZ-LUQUE, Sevilla (ES)

(73) Assignee: Adhera Health, Inc., Santa Cruz, CA (US)

(21) Appl. No.: 18/829,944

(22) Filed: Sep. 10, 2024

Related U.S. Application Data
(63) Continuation-in-part of application No. PCT/US2023/015031, filed on Mar. 11, 2023.
(60) Provisional application No. 63/269,247, filed on Mar. 11, 2022.

(51) Int. Cl. G16H 50/30; A61B 5/00; A61B 5/16; G16H 10/60; G16H 80/00

(52) U.S. Cl. CPC G16H 50/30; A61B 5/165; A61B 5/7275; G16H 10/60; G16H 80/00

(57) ABSTRACT

A method for the semantic understanding of subjective and objective health data in systems for supporting the self-management of a user with at least one chronic condition, said system providing health assessment to the user related to the at least one chronic condition, wherein said method at least involves using a first module for combining user's objective health data and user's subjective health data, a second module for producing an assessment to the user and a third module for generating an explanation to the user, the method carried out by at least one processor, said method comprising: a) receiving by the at least one processor a plurality of first health parameters of a user, wherein said first health parameters denote user's objective health data and wherein at least one of the first health parameters represents data measured by an external device; and b) receiving by the at least one processor a plurality of second health parameters of a user, wherein said second health parameters comprise at least one subjective health measurement; wherein said at least one subjective health measurement denotes a perception of the user's subjective health; and c) combining by the first module in the at least one processor the objective health data and the subjective health data; and d) producing by the second module in the at least one processor, at least one health related assessment to the user comprising content data, said assessment based on the combined objective and subjective health data; and wherein said producing uses artificial intelligence methods; and e) generating by the third module in the at least one processor, at least one explanation to the user, wherein said explanation is related to the produced assessment; and f) providing the at least one assessment and the at least one explanation to the user.

### RELATED APPLICATIONS

This application is a continuation-in-part of International Application No. PCT/US2023/015031 filed Mar. 11, 2023, which claims priority to U.S. Application No. 63/269,247 filed Mar. 11, 2022, each of which is hereby incorporated herein by reference in its entirety.

### FIELD OF THE INVENTION

The present invention relates generally to medical methods and systems for monitoring the health of people with chronic conditions, and more particularly to methods and systems for the semantic understanding of both subjective and objective health.

### BACKGROUND OF THE DISCLOSURE

Many health symptoms have a significant impact on the quality of life of people with chronic conditions that are modulated by subjective and physical health contributing factors. The assessment of those symptoms tends to rely always on a combination of objective and subjective health data. However, there is a lot of heterogeneity on how the assessment of those health symptoms is done which results in serious limitations for data-driven applications.

Across several therapeutic areas there are use cases of longitudinal data which includes different objective health data sources (e.g., polysomnography for a sleep study, actigraphy for studies of physical activity and sleep). These objective health data representations are not typically annotated with relevant subjective health data (e.g., perceived sleep quality based on a psychometric, stress, anxiety).

The understanding and quantification of health symptoms that have both an objective and subjective dimension are very difficult and consequently, there are challenges with regards to reliability, subjectivity in the clinical decision making. This is to a large extent due to the high variability across patients and also within the same patient health journey.

The prior art fails to teach a method that complements objective health measurements and engagement data by automatically capturing subjective health measurements after triggering ecological momentary assessments.

Without a dynamic and personalized capture and combination of objective health measurements with subjective health measurements, it would not be possible to produce a comprehensive context-driven personalization for patients within given digital health programs.

Prior art in patient monitoring fails to consider patient engagement data that would trigger subjective ecological momentary assessments to capture patient's contextual subjective health.

There is a need for ensuring the semantic integration and representation of objective and subjective measurements. This semantic integration and representation could be crucial to developing explainable artificial intelligence-based solutions in the health domain. For example, the subjective perception of sleep quality could enrich the objective sleep quality measured by a sensor or device (e.g., wearables).

There is a need to improve artificial intelligence-based health outcomes predictions in a way that increases their explainability and actionability to patients and clinicians. The prior art is limited due to the lack of integration of strategies that facilitate the explainability and actionability of those predictions to users.

As will be clearly understood from the next sections, the present invention discloses a method and system for assessing behavioral and mental health changes by automatically monitoring and modeling the patient's context.

### SUMMARY OF THE INVENTION

The embodiments disclosed herein are only examples of the many possible advantageous uses and implementations of the innovative teachings presented herein. In general, statements made in the specification of the present disclosure do not necessarily limit any of the various claimed inventions. Moreover, some statements may apply to some inventive features but not to others. In general, unless otherwise indicated, singular elements may be in plural and vice versa with no loss of generality. In the drawings, numerals refer to like parts through several views.

The term “mobile device” and similar, as used herein is intended to include but not be limited to any of the following: mobile telephone, smartphone, PDA, smartwatch, iPad, game console, tablet, laptop, or another computer terminal, and embedded remote unit.

The term “sensor” and similar, as used herein is intended to include but not be limited to any of the following: wearable sensors, audio sensors (e.g., microphone, an acoustic transducer, etc.), video sensor (e.g., video camera, IR camera, gesture sensor, etc.), weight sensor (e.g., scale, BMI sensor, physiological sensors, and implantable sensors.

The term “health data” and similar, as used herein is intended to include but not be limited to any of the following: Electronic health records, self-reported health data, therapeutic data, clinical data, psychological reports, psychiatric reports, behavioral and mental health data, health data provided by personal devices (e.g., wearable sensors), sensors that are used as “digital biomarker” of health-related measurements (e.g., depression assessment from wearables), etc. Health data provided by personal devices may be online data (i.e., reported in real-time) or offline (i.e., recorded data). The health data also includes demographics and other social-related factors such as education and cultural preferences. For example, the health data may include biopsychosocial models of data which consider the interconnection between biological, psychological, and socio-environmental factors (e.g., educational level, income level, neighborhood and mother tongue) specifically how these aspects play a role in health. The health data can be related to both the patients or/and their family caregivers. The health data can also include family interpersonal dynamics (e.g., communication issues between family caregiver and the patient).

The term “engagement data” and similar, as used herein is intended to include but not be limited to any of the following: the interactions of the users with the different elements of the digital solutions including ratings of content, views of content, and any other interactions with elements available in the user interface.

The term “user model” and similar, as used herein is intended to include but not be limited to any of the following: the combination of data that belongs to an individual and it is used for modeling proposes, that includes health data and engagement data. The user model can refer to both the patients with the chronic conditions or their family caregivers who are users of this invention.

The term “health meta-feature”, as used herein is intended to include but not be limited to any of the following: meta-data which describe characteristics of therapeutic and sensing content with regards to their design towards addressing aspects related to particular behavioral and mental health factors (e.g., content designed for users with low mood) and also physical health (e.g., content designed for users with a particular type of surgery). These meta-features also address related social health determinant factors when relevant. These meta-features combined represent a semantic space and a taxonomy of the contents of the Health Recommender System.

The term “health-gram”, as used herein is intended to include but not be limited to any of the following: the longitudinal representation of all health data related to the users, including, among others, the subjective health data captured subjectively by asking the user (e.g., the question “how do you feel today?”) and objective health data (e.g., number of steps) that can come from both sensors and other relevant data sources (e.g., prescribed medications).

The term “recommendation” and similar, as used herein is intended to include but not be limited to any of the following: recommendations of therapeutic content which are tailored to the individual unique health needs and aiming at supporting the self-management of the patients, including both mental and physical health aspects such as lifestyle recommendations, behavioral and emotional support. The recommendations may also include sensing content (e.g., quizzes and psychometrics) that are provided to users at the right time and place. Recommendations may also include both the selection for the right content and also its adaptation (e.g., modification of the content to further personalize using specific patient's data).

The term “objective measurement” or “objective data” or “objective health data” and similar, as used herein is intended to include but not be limited to any of the following: measurements that are not influenced by the opinion or perspective of people, such as age, lab value results, and sensor data. Objective health data may also comprise information extracted from EMR (electronic medical records) as provided by medical systems.

The term “subjective measurement” or “subjective data” or “subjective health data” and similar, as used herein is intended to include but not be limited to any of the following: measurements that are inherently depending on the perspective of the person making the measurement, that includes psychometrics such as perceived tiredness or mood.

The term “ecological momentary assessment” refers to a subtype of assessment in real-time where users report on their current behaviors and experience at the right place (aka, ecological context), and the right time (aka momentary context).

The term “therapeutic content” and similar, as used herein is intended to include but not be limited to any of the following: refers to digital content that has been designed to provide a therapeutic value (aka supporting patient self-management), that therapeutic content might include motivational messages, mental wellbeing exercises, educational content among others. The therapeutic content does contain metadata with relevant health meta-features. The content model includes both the content itself (e.g., educational video) and the metadata describing the health and behavioral meta-features which define the characteristics for which the content is used (e.g., whether the content is designed for low mood patients or family caregivers).

The term “semantic integration” and similar, as used herein is intended to include but not be limited to any of the following: to the integration of data in a way that its meaning is annotated to facilitate the integration of heterogeneous data sources of related meanings.

The term “Ecological Momentary Assessment” and similar, as used herein is intended to include but not be limited to any of the following: to psychometrics (also referred to as questionnaires) that are asked to a user at the right context, which includes at the right location/ecological and temporal/momentary.

The present invention here described, provides computer-based medical systems and methods for monitoring the health of people affected by chronic conditions including both patients and their family caregivers. In particular, the invention is related to methods and systems for the semantic understanding of subjective and objective health that may be required to support the self-management of symptoms of chronic conditions by automatic monitoring and modeling the patient's context.

The assessment of the physical and mental health of the patients may rely on the semantic integration of objective and subjective health data. The subjective health data (e.g., psychometrics, quizzes for knowledge evaluation) may be captured to enrich the objective health data coming from sensors and other clinical data sources. The disclosed system may include a module that based on the context of the patient does dynamically trigger events to capture subjective health data (e.g., launching a short psychometric after doing physical activity detected by a sensor). This subjective health data may provide a longitudinal multi-dimensional representation of the wellbeing of the patient which then can be used by different data-driven applications to support patients and healthcare professionals.

For example, Chronic condition-related fatigue (CrF) is one of the most common symptoms affecting people with chronic conditions such as cancer or multiple sclerosis. Fatigue, the feeling of being very tired that may affect the functioning of patients, may be a major health problem that may be linked to a range of contributing factors (i.e., mood, sleep, physical activity). The assessment of those factors is traditionally done with questionnaires and other clinical procedures in the clinic (i.e., interviews, lab tests).

According to an aspect of the present disclosure, there is provided a computer implemented method for the semantic understanding of subjective and objective health data in systems for supporting the self-management of a user (e.g., patient) with at least one chronic condition, said system providing health assessment to the user related to the at least one chronic condition, wherein said method at least involves using a first module for combining user's objective health data and user's subjective health data, a second module for producing an assessment to the user and a third module for generating an explanation to the user, the method carried out by at least one processor, said method comprising: a) receiving by the at least one processor a plurality of first health parameters of a user, wherein said first health parameters denote user's objective health data and wherein at least one of the first health parameters represents data measured by an external device; and b) receiving by the at least one processor a plurality of second health parameters of a user, wherein said second health parameters comprise at least one subjective health measurement; wherein said at least one subjective health measurement denotes a perception of the user's subjective health; and c) combining by the first module in the at least one processor the objective health data and the subjective health data; and d) producing by the second module in the at least one processor, at least one health related assessment to the user comprising content data, said assessment based on the combined objective and subjective health data; and wherein said producing uses artificial intelligence methods; and e) generating by the third module in the at least one processor, at least one explanation to the user, wherein said explanation is related to the produced assessment; and f) providing the at least one assessment and the at least one explanation to the user.

As may be apparent to one skilled in the art, the number of modules implementing the method and the functions performed by each of them may vary and all are consistent with the embodiments of the present disclosure. For example, in some embodiments, the modules may consist of separate SW modules (i.e., in some cases even executed by different processors) while in other embodiments the division to modules is only functional.

According to an embodiment of one or more paragraph(s) of this disclosure, the combination of data listed above may be used to create a health-gram which is a graphical representation of the physical and mental health of the user. In some embodiments, in the case of fatigue, that may further include time-based data.

According to an embodiment of one or more paragraph(s) of this disclosure, the at least one processor is operative to communicate with at least one external device (e.g., smartphone, wearable device, tablet, personal computer, wired and/or wireless sensor, and the like) and wherein said external device is operable to exchange data with the at least one processor. According to some embodiments, the objective health data may be received from at least one sensor or clinical data set.

According to an embodiment of one or more paragraph(s) of this disclosure, the subjective health data may comprise at least one of psychometric parameters, quizzes for knowledge evaluation, and the like. In some embodiments, the subjective health data may be used to enrich the objective health data. According to some embodiments, the subjective health data may at least comprise semantic integration of the subjective health data captured in a digital health solution with explicit user feedback.

According to an embodiment of one or more paragraph(s) of this disclosure, the processor may have further access to previous assessments provided by the system (e.g., assessments provided to the same user in the past, assessments provided to other users with similar physical and/or mental health conditions, etc.) and wherein producing by the second module in the at least one processor, the at least one health related assessment to the user may also comprise the interaction of the patient with at least one previous assessment.

According to an embodiment of one or more paragraph(s) of this disclosure, this assessment may be related to the physical and mental health of the user and may rely on the semantic integration of objective and subjective health data.

According to an embodiment of one or more paragraph(s) of this disclosure, the method may further involve a fourth module that based on the context of the patient, may dynamically trigger at least one event to capture additional subjective health data. According to some embodiments, the additional subjective health data may be used to complement and increase the quality of the subjective health data and engagement data. According to some embodiments, at least one event may comprise launching a psychometric (i.e., ecological momentary assessment), wherein said psychometric may be performed after a physical activity of the user or after a sleep period of the user. According to some embodiments, the system may receive a notification for such an event from a wearable sensor (e.g., worn by the user). According to some embodiments, scheduling the trigger of at least one event may be based on methods that dynamically adjust the number of triggers based on the user's engagement data constraints.

According to an embodiment of one or more paragraph(s) of this disclosure, the method may produce a comprehensive context-driven personalization for patients based on the dynamic and personalized capture and combination of objective health measurements with subjective health measurements. According to some embodiments, the method may ensure the semantic integration and representation of objective and subjective measurements. As may be apparent to one skilled in the art, this semantic integration and representation may be crucial to develop explainable artificial intelligence-based solutions in the health domain. According to some embodiments, the method may improve artificial intelligence-based health outcomes predictions in a way that may increase their explainability and actionability to patients and clinicians.

According to an embodiment of one or more paragraph(s) of this disclosure, the method considers patient engagement data that may trigger subjective ecological momentary assessments to capture patient's contextual subjective health.

According to an embodiment of one or more paragraph(s) of this disclosure, the method may provide at least one assessment and at least one explanation tailored to the personal characteristics of the user (e.g., the maximum number of assessments per day, the language of the assessment and/or explanation, users' preferences, etc.).

As may be apparent to one skilled in the art, combining the objective health data and the subjective health data to produce an assessment may be performed in many ways and using different algorithms. According to some embodiments, said producing may comprise adapting at least one assessment to the user, by combining artificial intelligence models about symptoms predicted trends and their contributing factors. According to some embodiments, the first and/or second health parameters may further comprise weighing factors and wherein said producing may further consider said weighing factors to produce at least one assessment.

According to an embodiment of one or more paragraph(s) of this disclosure, the computer implemented method for the semantic understanding of subjective and objective health data may further comprise an approach for the characterization of fatigue, wherein producing an assessment further applies software that provides insights (e.g., to clinicians and/or patients) that support the management of fatigue. This may comprise:
Objective health data: diagnostic and health data coming from sensors (i.e., wearables, voice, etc.)
Subjective health data:
   Psychometrics: traditional psychometrics, ecological momentary assessment, and adaptative testing.
   Therapeutic content engagement: rating and quizzes on content that provides knowledge and preference clues of the patient.

According to an embodiment of one or more paragraph(s) of this disclosure, the computer implemented method for the semantic understanding of subjective and objective health data may further make use of health-grams which may be applied to create explainable machine learning solutions that may support both patients and clinicians.

According to another aspect of the present disclosure, there is provided a system, operative to support the self-management of people with at least one chronic condition, and to provide assessments related to their psychical and mental health, the system comprising at least one processor comprising at least one communication channel configured to communicate with external devices to receive and store a plurality of first health parameters of a user, wherein said first health parameters denote user's objective health data and wherein at least one of the first health parameters represents data measured by an external device; and wherein said at least one processor is further configured receive a plurality of second health parameters of a user, wherein said second health parameters comprise at least one subjective health measurement; wherein said at least one subjective health measurement denotes a perception of the user's subjective health; and access to a non-transitory computer readable storage medium used to store and retrieve the plurality of first health parameters, the plurality of second health parameters, and additional data; wherein the at least one processor is configured to perform a computer implemented method for the semantic understanding of the subjective and the objective health data, wherein said method at least involve using a first module for combining user's objective health data and user's subjective health data, a second module for producing an assessment to the user and a third module for generating an explanation to the user, said method comprising: a) receiving by the at least one processor a plurality of first health parameters of a user, wherein said first health parameters denote user's objective health data and wherein at least one of the first health parameters represents data measured by an external device; and b) receiving by the at least one processor a plurality of second health parameters of a user, wherein said second health parameters comprise at least one subjective health measurement; wherein said at least one subjective health measurement denotes a perception of the user's subjective health; and c) combining by the first module in the at least one processor the objective health data and the subjective health data; and d) producing by the second module in the at least one processor, at least one health related assessment to the user comprising content data, said assessment based on the combined objective and subjective health data; and wherein said producing uses artificial intelligence methods; and e) generating by the third module in the at least one processor, at least one explanation to the user, wherein said explanation is related to the produced assessment; and f) providing the at least one assessment and the at least one explanation to the user.
a. According to another aspect of the present disclosure, there is provided a non-transitory computer readable storage medium in a system operative to support the self-management of people with at least one chronic condition, and to provide assessments related to their psychical and mental conditions, the system comprising at least one processor comprising at least one communication channel configured to communicate with external devices to receive and store a plurality of first health parameters of a user, wherein said first health parameters denote user's objective health data and wherein at least one of the first health parameters represents data measured by an external device;
   and wherein said at least one processor is further configured receive a plurality of second health parameters of a user, wherein said second health parameters comprise at least one subjective health measurement; wherein said at least one subjective health measurement denotes a perception of the user's subjective health; and wherein said computer readable storage medium stores at least one readable program, and when said at least one readable program is executed by the at least one processor causes the at least one processor to perform a computer implemented method for the semantic understanding of the subjective and the objective health data, wherein said method at least involve using a first module for combining user's objective health data and user's subjective health data, a second module for producing an assessment to the user and a third module for generating an explanation to the user, said method comprising: a) receiving by the at least one processor a plurality of first health parameters of a user, wherein said first health parameters denote user's objective health data and wherein at least one of the first health parameters represents data measured by an external device; and b) receiving by the at least one processor a plurality of second health parameters of a user, wherein said second health parameters comprise at least one subjective health measurement; wherein said at least one subjective health measurement denotes a perception of the user's subjective health; and c) combining by the first module in the at least one processor the objective health data and the subjective health data; and d) producing by the second module in the at least one processor, at least one health related assessment to the user comprising content data, said assessment based on the combined objective and subjective health data; and wherein said producing uses artificial intelligence methods; and e) generating by the third module in the at least one processor, at least one explanation to the user, wherein said explanation is related to the produced assessment; and f) providing the at least one assessment and the at least one explanation to the user.

One skilled in the art will realize the invention may be embodied in other specific forms without departing from the spirit or essential characteristics thereof. The foregoing embodiments are therefore to be considered in all respects illustrative rather than limiting of the invention described herein. In the foregoing detailed description, numerous specific details are set forth in order to provide an understanding of the invention. However, it will be understood by those skilled in the art that the invention can be practiced without these specific details. In other instances, well-known methods, procedures, components, modules, units, and/or circuits have not been described in detail so as not to obscure the invention. Some features or elements described with respect to one embodiment can be combined with features or elements described with respect to other embodiments. The embodiments referred to above, and other embodiments, are described in detail in the next section.

### BRIEF DESCRIPTION OF THE DRAWINGS

The subject matter that is regarded as the invention is particularly pointed out and distinctly claimed in the claims at the conclusion of the specification. The foregoing and other objects, features, and advantages of the invention will be apparent from the following detailed description taken in conjunction with the accompanying drawings.

FIG. 1 is a schematic diagram showing the main elements of a system comprising the Ecological Momentary Assessment Engine which relies on the inputs/changes in the user models in accordance with an exemplary embodiment of the present invention.

FIG. 2 is a schematic diagram showing an exemplary embodiment of the flow of triggering an Ecological Momentary Assessment output based on Objective Sensor Data in accordance with an exemplary embodiment of the present invention.

FIG. 3 is a schematic diagram showing another exemplary embodiment of the flow of triggering an Ecological Momentary Assessment output based on Objective Sensor Data in accordance with an exemplary embodiment of the present invention.

FIG. 4 is a schematic diagram showing the main elements of the health trends prediction process and system in accordance with an exemplary embodiment of the present invention.

FIG. 5 is a schematic diagram showing the user interface showing the outputs of the Machine Learning Health Trends Predictor and the Explainer Module Data in accordance with an exemplary embodiment of the present invention.

### DETAILED DESCRIPTION OF THE INVENTION

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise.

It will be further understood that the terms “comprises” and/or “comprising” and/or “includes” and/or “including” when used in this specification, specify the presence of stated features, steps, operations, elements, and/or components, but do not preclude the presence or addition of one or more other features, steps, operations, elements, components, and/or groups thereof.

Unless otherwise defined, all terms (including technical and scientific terms) used herein have the same meaning as commonly understood by one having ordinary skill in the art to which this invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

In describing embodiments of the invention, it will be understood that a number of techniques and steps are disclosed. Each of these has individual benefit and each can also be used in conjunction with one or more, or in some cases all, of the other disclosed techniques. Accordingly, for the sake of clarity, this description will refrain from repeating every possible combination of the individual steps in an unnecessary fashion.

Nevertheless, the specification and claims should be read with the understanding that such combinations are entirely within the scope of the invention.

In the following description, for purposes of explanation, numerous specific details are set forth in order to provide a thorough understanding of the present invention. It will be evident, however, to one skilled in the art that the present invention may be practiced without these specific details.

The present disclosure is to be considered as an exemplification of the invention and is not intended to limit the invention to the specific embodiments illustrated by the figures or description below.

Reference will now be made in detail to the present embodiments of the disclosure, certain examples of which are illustrated in the accompanying drawings.

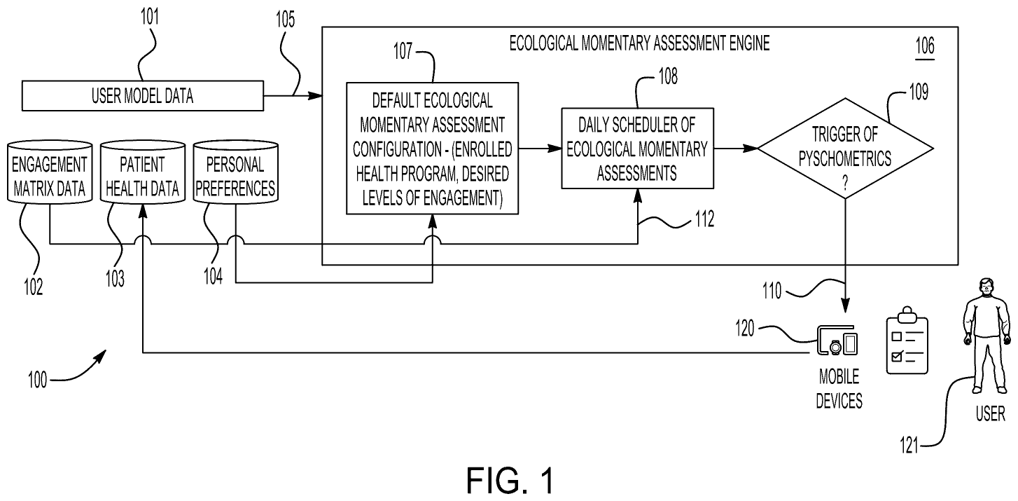

FIG. 1 is a schematic diagram illustrating an exemplary embodiment of the main elements of a system 100 comprising the ecological momentary assessment engine 106 which relies on the inputs/changes in the user models 101 that may be used to implement certain aspects of the disclosed embodiments. The components and elements that are illustrated in FIG. 1 may vary consistent with the disclosed embodiments.

In one embodiment, the main elements of the system 100 comprising the ecological momentary assessment engine 106 may include the inputs/changes derived in a step 105 from the user models 101. In some embodiments, the user models 101 are based on personal preferences 104, engagement matrix data 102, and patient health data 103. The ecological momentary assessment engine 106 includes a default configuration 107 per health program which is received in a step 111 from the personal preferences 111. According to some embodiments, the default configuration 107 may be used as input in the daily scheduler 108 of momentary ecological assessments, which also may use as input data in the user model 101 including engagement matrix data 102 and patient health data 103.

According to some embodiments, the daily scheduler 108 may be based on different methods, including methods that dynamically adjust the number of triggers based on patient engagement data constraints (e.g., maximum five ecological momentary assessments per day) or more advanced methods (e.g., artificial intelligence-based methods such as reinforcement that aims to optimize response rate). The daily scheduler 108 may initiate a trigger 109 of the psychometrics/ecological momentary assessment in the right context.

According to some embodiments, the interaction of the user with the psychometric via mobile devices 120 may create in a step 122 a new log into the health data profile 103 of the user model data 101.

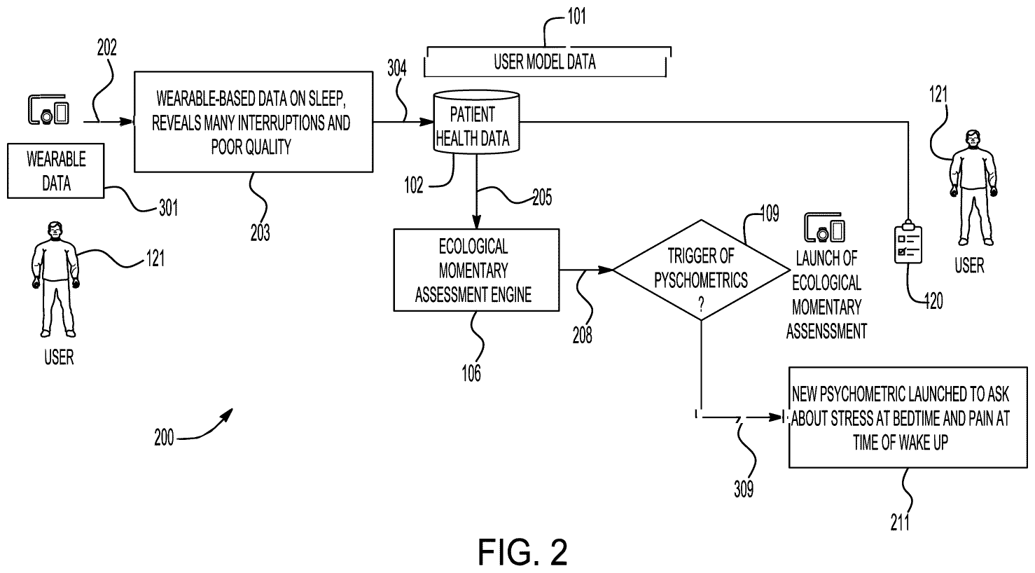

FIG. 2 is a schematic diagram illustrating an exemplary embodiment of a method 200 of triggering an ecological momentary assessment output based on objective sensor data. The components and elements that are illustrated in FIG. 2 may vary consistent with the disclosed embodiments.

In one embodiment, the method 200 of triggering an ecological momentary assessment output 210, 211 based on objective sensor data may start from a wearable sensor 201 reporting in a step 202 a report 203 of poor sleep quality of a user 121. This report 203 may consequently update in a step 204 the patient health data 103 of the user model 101. The ecological momentary assessment engine 106 may take in a step 205 the patient health data 103 and schedule in a step 208 a psychometric 211 which gets triggered in a step 209 when the user 121 is about to go to sleep to ask about pain and stress, since those are known factors of poor sleep quality.

According to some embodiments, launching the ecological momentary assessment in a step 210 may be performed by sending a message (e.g., SMS, WhatsApp, etc.) to the user's mobile phone 120.

According to some embodiments, after the user 121 completes answering the psychometric 211 in the mobile device 120, the result may be transmitted back to the system 206 and then stored in the patient health data 103.

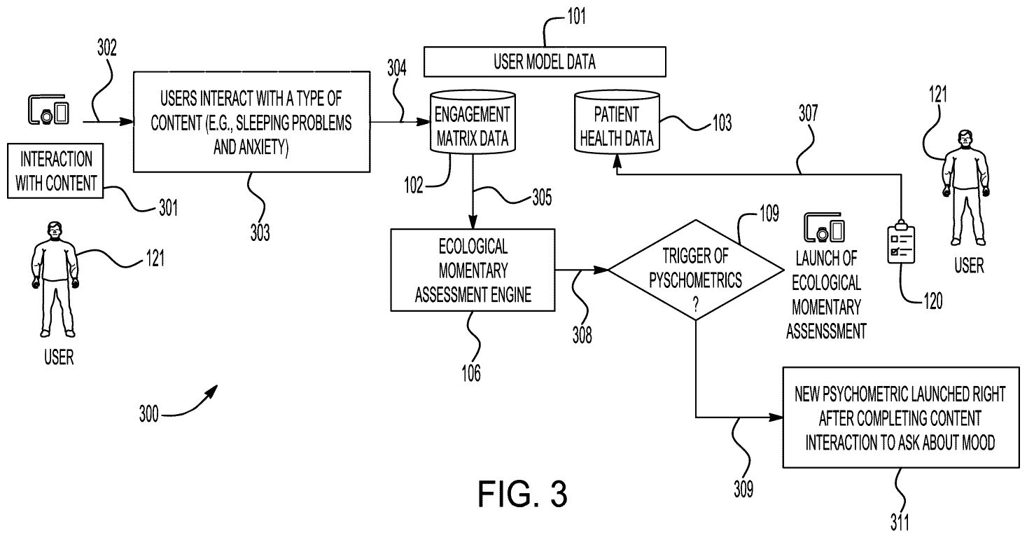

FIG. 3 is a diagram illustrating another exemplary embodiment of a method 300 of triggering an ecological momentary assessment output based on objective sensor data. The components, and elements that are illustrated in FIG. 3 may vary consistent with the disclosed embodiments.

According to some embodiments, method 300 depicts the triggering step 305 of the ecological momentary assessment 106 as a result of the user 121 interacting with a particular type of content (e.g., sleeping problem and anxiety) 303 that may result in an update step 304 of the engagement matrix data 102 of the user model 101. According to some embodiments, this update step 304 is detected in a step 305 by the ecological momentary assessment engine 106. According to some embodiments, this update 304 step initiates in a step 308 the trigger 109 of psychometrics 311 that may launch an ecological momentary assessment 210 to evaluate the mood of the user 121.

According to some embodiments, after the user 121 responds to the ecological momentary assessment 210, for example using his/her mobile device 120, the response is then transmitted in a step 307 back to the system and then stored in the patient health data 103.

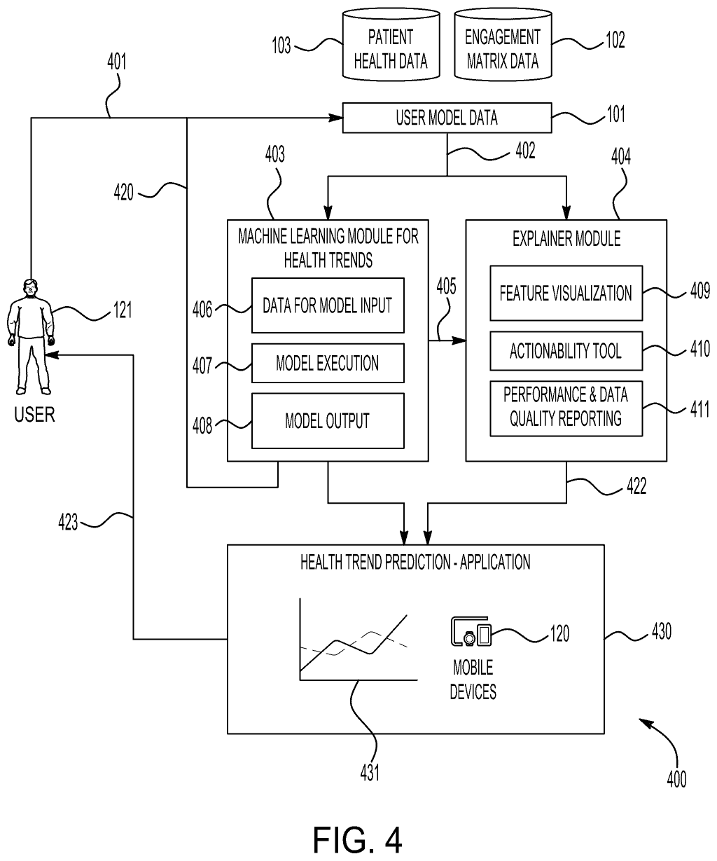

FIG. 4 is a diagram illustrating an exemplary embodiment of the main elements of the health trends prediction process and system 400. The components and elements that are illustrated in FIG. 4 may vary consistent with the disclosed embodiments.

According to some embodiments, the user model data 101 (including patient health data 103, and engagement matrix data 102) may be the data inputs 402 for the a) machine learning module for health trend prediction 403 and b) explainer module 404. The machine learning module for health trend prediction 403 includes a model execution 407 for processing the user data 101 to be used as input 406 of the already trained machine learning modules (e.g., deep learning models, and any other artificial intelligence model), then the output is generated in a model output 408. The explainer module relies on both the data inputs 402 from the user model 101 and also different characteristics of the model and its output 405, such as the predictive weight of each variable/feature used in the model (feature visualizations 409) (e.g., SHAP technique, Shapley Additive explanations. A SHAP analysis of a model will indicate how significant each factor is in determining the final prediction that the model outputs.), actionability tools 410 (e.g., what-if-techniques showing what would have changed outcomes), algorithmic performance data 411 (e.g., AUC, Specificity). The outputs 422, 421 of both the explainer module 404 and the machine learning module for health trends 403 are then visualized in the user interfaces 430, of both clinicians 431 and patients 120 depending on the use case.

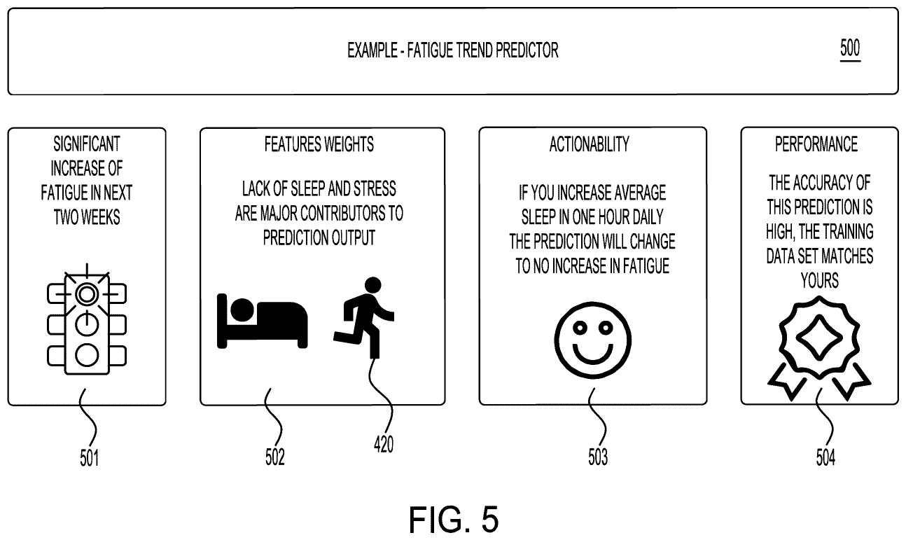

FIG. 5 is a diagram illustrating an exemplary embodiment of the user interface showing the outputs of the machine learning health trends predictor and the explainer module 500. The components and elements that are illustrated in FIG. 5 may vary consistent with the disclosed embodiments.

According to some embodiments, the user interface showing the outputs of the machine learning health trends predictor and the explainer module may be used as a fatigue trend predictor 500. According to some embodiments, the fatigue trend predictor 500 may be suitable for patients with cancer.

For example, the system shows: a) increased right of higher fatigue in the next following two weeks 501, b) which features are more positively affecting that prediction (i.e., stress and poor sleep quality) 502, c) actionability output showing which modifiable variables might have change the prediction (i.e., increased sleep time) 503, d) a summary of the reliability of the prediction and the quality of the data 504. According to some embodiments, these outputs may be used to provide motivational content to patients to better manage fatigue symptoms or enhance the understanding and evaluation of fatigue by clinicians.

The principles of the invention, wherever applicable, are implemented as hardware, firmware, software, or any combination thereof. Moreover, the software is preferably implemented as an application program tangibly embodied on a program storage unit or computer-readable medium. The application program may be uploaded to and executed by, a machine comprising any suitable architecture. Preferably, the machine is implemented on a computer platform having hardware such as one or more central processing units (“CPUs”), a memory, and input/output interfaces. The computer platform may also include an operating system and microinstruction code. The various processes and functions described herein may be either part of the microinstruction code or part of the application program, or any combination thereof, which may be executed by a CPU, whether or not such computer or processor is explicitly shown. In addition, various other peripheral units may be connected to the computer platform such as an additional data storage unit and a printing unit. The circuits described hereinabove may be implemented in a variety of manufacturing technologies well known in the industry including but not limited to integrated circuits (ICs) or SIP (system on chip) and discrete components that are mounted using surface mount technologies (SMT), and other technologies.

The system of the present invention may include, according to certain embodiments of the invention, machine-readable memory containing or otherwise storing a program of instructions which, when executed by the machine, implements some or all of the system, methods, features, and functionalities of the invention shown and described herein. Alternatively, or in addition, the system of the present invention may include, according to certain embodiments of the invention, a program as above which may be written in any conventional programming language, and optionally a machine for executing the program such as but not limited to a general-purpose computer which may optionally be configured or activated in accordance with the teachings of the present invention. Any of the teachings incorporated herein may, wherever suitable, operate on signals representative of physical objects or substances.

The term “program” or “computer program” may include computer program code means for performing any of the methods shown and described herein when said program is run on at least one computer; and a computer program product, comprising a typically non-transitory-computer-usable or computer-readable medium, typically tangible, having a computer-readable program code embodied therein, said computer-readable program code adapted to be executed to implement any or all of the methods shown and described herein. Computer program code may be written in any suitable programming language and may be executed on a single computer system, or a plurality of computer systems. The operations in accordance with the teachings herein may be performed by at least one computer specially constructed for the desired purposes or a general-purpose computer specially configured for the desired purpose by at least one computer program stored in a typically non-transitory computer-readable storage medium. The term “non-transitory” is used herein to exclude transitory, propagating signals or waves, but to otherwise include tangible items including any volatile or non-volatile computer memory technology suitable to the application.

Any suitable processor/s, display, and input means may be used to process, display e.g., on a computer screen or other computer output device, store, and accept information such as information used by or generated by any of the methods and systems that are shown and described herein: the above processor/s, display, and input means including computer programs, in accordance with some or all of the embodiments of the present invention. Any or all functionalities of the invention shown and described herein, such as but not limited to operations within flowcharts, may be performed by anyone or more of: at least one conventional personal computer processor, workstation, or other programmable device or computer or electronic computing device or processor, either general-purpose or specifically constructed, used for processing; a computer display screen and/or printer and/or speaker for displaying; machine-readable memory such as optical disks, CDROMs, DVD, Blu-ray, magnetic-optical discs or other discs; RAMs, ROMs, EPROMS, EEPROMs, Flash memories, magnetic or optical or other cards, for storing, and keyboard or mouse for accepting. Modules shown and described herein may include any one or combination or a plurality of a server, a data processor, a memory/computer storage, a communication interface, a computer program stored in memory/computer storage.

The term “process” as used in this disclosure is intended to include any type of computation or manipulation or transformation of data (e.g., health data) represented as physical, e.g., electronic, phenomena which may occur or reside e.g., within registers and/or memories of at least one computer or processor. The term processor includes a single processing unit or a plurality of distributed or remote such units.

Any trademark occurring in the text or drawings is the property of its owner and occurs herein merely to explain or illustrate one example of how an embodiment of the invention may be implemented.

Unless specifically stated otherwise, as apparent from the following discussions, it is appreciated that throughout the specification discussions, utilizing terms such as, “processing”, “computing”, “estimating”, “selecting”, “calculating”, “determining”, “generating”, “reassessing”, “classifying”, “generating”, “producing”, “registering”, “detecting”, “associating”, “obtaining” or the like, refer to the action and/or processes of at least one computer/s or computing system/s, or processor/s or similar electronic computing device/s, that manipulate and/or transform data represented as physical, such as electronic, quantities within the computing system's registers and/or memories, into other data similarly represented as physical quantities within the computing system's memories, registers or other such information storage, transmission or display devices. The term “computer” or “processor” should be broadly construed to cover any kind of electronic device with data processing capabilities, including, by way of non-limiting example, personal computers, servers, a computing system, communication devices, processors (e.g., digital signal processor (DSP), microcontrollers, field-programmable gate array (FPGA), application-specific integrated circuit (ASIC), etc.) and other electronic computing devices.

The present invention may be described, merely for clarity, in terms of terminology specific to particular programming languages, operating systems, browsers, system versions, individual products, and the like. It will be appreciated that this terminology is intended to convey general principles of operation clearly and briefly, by way of example, and is not intended to limit the scope of the invention to any particular programming language, operating system, browser, system version, or individual product.

Elements separately listed herein need not be distinct components and alternatively may be the same structure. A statement that an element or feature may exist is intended to include (a) embodiments in which the element or feature exists; (b) embodiments in which the element or feature does not exist; and (c) embodiments in which the element or feature exist selectively (e.g., a user may configure or select whether the element or feature does or does not exist). Any suitable input device, such as but not limited to a sensor, may be used to generate or otherwise provide information received by the apparatus and methods shown and described herein.

All examples and conditional language recited herein are intended for pedagogical purposes to aid the reader in understanding the principles of the invention and the concepts contributed by the inventor to furthering the art and are to be construed as being without limitation to such specifically recited examples and conditions. Moreover, all statements herein reciting principles, aspects, and embodiments of the invention, as well as specific examples thereof, are intended to encompass both structural and functional equivalents thereof. Additionally, it is intended that such equivalents include both currently known equivalents as well as equivalents developed in the future, i.e., any elements developed that perform the same function, regardless of structure.

1. A method for the semantic understanding of subjective and objective health data in systems for supporting the self-management of a user with at least one chronic condition, said system providing health assessment to the user related to the at least one chronic condition, wherein said method at least involves using a first module for combining user's objective health data and user's subjective health data, a second module for producing an assessment to the user and a third module for generating an explanation to the user, the method carried out by at least one processor, said method comprising:
a) receiving by the at least one processor a plurality of first health parameters of a user, wherein said first health parameters denote user's objective health data and wherein at least one of the first health parameters represents data measured by an external device; and
b) receiving by the at least one processor a plurality of second health parameters of a user, wherein said second health parameters comprise at least one subjective health measurement; wherein said at least one subjective health measurement denotes a perception of the user's subjective health; and
c) combining by the first module in the at least one processor the objective health data and the subjective health data; and
d) producing by the second module in the at least one processor, at least one health related assessment to the user comprising content data, said assessment based on the combined objective and subjective health data; and wherein said producing uses artificial intelligence methods; and
e) generating by the third module in the at least one processor, at least one explanation to the user, wherein said explanation is related to the produced assessment; and
f) providing the at least one assessment and the at least one explanation to the user.

2. The method of claim 1 wherein said at least one processor is configured to communicate with at least one external device and wherein said external device is configured to exchange data with the at least one processor.

3. The method of claim 2 wherein said objective health data is received from at least one of sensor and clinical data set.

4. The method of claim 3 wherein said subjective health data comprises at least one of psychometric parameters, quizzes for knowledge evaluation, and the like.

5. The method of claim 4 wherein said subjective health data is used to enrich the objective health data.

6. The method of claim 1 wherein said subjective health data at least comprises semantic integration of the subjective health data captured in a digital health solution with explicit user's feedback.

7. The method of claim 1 wherein said processor has further access to previous assessments provided by the system and wherein said producing further comprises the interaction of the patient with at least one previous assessment.

8. The method of claim 1 wherein the at least one assessment is related to the physical and mental health of the user and relies on the semantic integration of objective and subjective health data.

9. The method of claim 1 wherein the method further involves a fourth module that based on the context of the patient dynamically triggers at least one event to capture additional subjective health data, said additional subjective health data used to complement and increase the quality of the subjective health data.

10. The method of claim 9 wherein said at least one event comprises launching a psychometric, said psychometric performed after at least one of physical activity of the user and a sleep period of the user.

11. The method of claim 9 wherein scheduling the trigger of the at least one event is based on methods that dynamically adjust the number of triggers based on user's engagement data constraints.

12. The method of claim 1, wherein said providing further comprises tailoring the at least one assessment and the at least one explanation to the personal characteristics of the user.

13. The method of claim 1 wherein said producing further comprises adapting the at least one assessment to the user, by combining artificial intelligence models about symptoms predicted trends and their contributing factors.

14. The method of claim 1, wherein said first and second health parameters further comprise weighing factors and wherein said producing further considers said weighing factors to produce the at least one assessment.

15. The method of claim 1, wherein the method further comprises an approach for the characterization of fatigue, wherein said producing further applies software that provides insights that support the management of fatigue.

16. A system, configured to support the self-management of people with at least one chronic condition, and to provide assessments related to their psychical and mental health, the system comprising:
at least one processor comprising at least one communication channel configured to communicate with external devices to receive and store a plurality of first health parameters of a user, wherein said first health parameters denote user's objective health data and wherein at least one of the first health parameters represents data measured by an external device;
and wherein said at least one processor is further configured receive a plurality of second health parameters of a user, wherein said second health parameters comprise at least one subjective health measurement; wherein said at least one subjective health measurement denotes a perception of the user's subjective health; and
a non-transitory computer readable storage medium used to store and retrieve the plurality of first health parameters, the plurality of second health parameters, and additional data;
wherein the at least one processor is configured to perform a computer implemented method for the semantic understanding of the subjective and the objective health data, wherein said method at least involve using a first module for combining user's objective health data and user's subjective health data, a second module for producing an assessment to the user and a third module for generating an explanation to the user, said method comprising:
a. receiving by the at least one processor a plurality of first health parameters of a user, wherein said first health parameters denote user's objective health data and wherein at least one of the first health parameters represents data measured by an external device; and
b. receiving by the at least one processor a plurality of second health parameters of a user, wherein said second health parameters comprise at least one subjective health measurement; wherein said at least one subjective health measurement denotes a perception of the user's subjective health; and
c. combining by the first module in the at least one processor the objective health data and the subjective health data; and
d. producing by the second module in the at least one processor, at least one health related assessment to the user comprising content data, said assessment based on the combined objective and subjective health data; and wherein said producing uses artificial intelligence methods; and
e. generating by the third module in the at least one processor, at least one explanation to the user, wherein said explanation is related to the produced assessment; and
f. providing the at least one assessment and the at least one explanation to the user.

17. A non-transitory computer readable storage medium in a system configured to support the self-management of people with at least one chronic condition, and to provide assessments related to their psychical and mental conditions, the system comprising:
at least one processor comprising at least one communication channel configured to communicate with external devices to receive and store a plurality of first health parameters of a user, wherein said first health parameters denote user's objective health data and wherein at least one of the first health parameters represents data measured by an external device;
and wherein said at least one processor is further configured receive a plurality of second health parameters of a user, wherein said second health parameters comprise at least one subjective health measurement; wherein said at least one subjective health measurement denotes a perception of the user's subjective health; and
wherein said computer readable storage medium stores at least one readable program, and when said at least one readable program is executed by the at least one processor causes the at least one processor to perform a computer implemented method for the semantic understanding of the subjective and the objective health data, wherein said method at least involve using a first module for combining user's objective health data and user's subjective health data, a second module for producing an assessment to the user and a third module for generating an explanation to the user, said method comprising:
a. receiving by the at least one processor a plurality of first health parameters of a user, wherein said first health parameters denote user's objective health data and wherein at least one of the first health parameters represents data measured by an external device; and
b. receiving by the at least one processor a plurality of second health parameters of a user, wherein said second health parameters comprise at least one subjective health measurement; wherein said at least one subjective health measurement denotes a perception of the user's subjective health; and
c. combining by the first module in the at least one processor the objective health data and the subjective health data; and
d. producing by the second module in the at least one processor, at least one health related assessment to the user comprising content data, said assessment based on the combined objective and subjective health data; and wherein said producing uses artificial intelligence methods; and
e. generating by the third module in the at least one processor, at least one explanation to the user, wherein said explanation is related to the produced assessment; and
f. providing the at least one assessment and the at least one explanation to the user.
